(19) United States
(12) Patent Application Publication
(10) Pub. No.: US 2008/0206849 A1
(43) Pub. Date: Aug. 28, 2008

(54) Assay Device & Method

(75) Inventors: Krzysztof Wojciech Zak, Bedford (GB); David Edward Williams, St. Heliers (NZ)

Correspondence Address: FOLEY HOAG, LLP;PATENT GROUP (w/ISA), 155 SEAPORT BLVD., BOSTON, MA, 02210-2600, US

(73) Assignee: Inverness Medical Switzerland GmbH, Zug (CH)

(21) Appl. No.: 11/573,319

(22) PCT Filed: Jul. 27, 2005
(86) PCT No.: PCT/GB2005/002927

§ 371 Date: Apr. 24, 2008

(30) Foreign Application Priority Data
Aug. 6, 2004 (GB) 0417601.2

(51) Int. Cl. C12M 1/00

(52) U.S. Cl. 435/287.2

(57) ABSTRACT

There is disclosed a lateral flow assay device and method of producing the same. The device, which is for identifying carbohydrate antigens in a biological sample comprises a substrate having, a) a sample receiving zone, b) an extraction zone for receiving the sample from said sample receiving zone and which extraction zone comprises immobilised or otherwise absorbed therein at least one or more reactants and reagents which when combined react to form an extraction reagent for a desired antigen in said sample, said sample to be added to said sample receiving zone optionally comprising the remaining reactant required to form said extraction reagent if not present in said extraction zone, c) optionally a neutralising agent capable of bringing the pH of the resulting sample to within the operational pH range of the assay and, d) a detection zone for a labelled specific binding or capture reagent for said antigen to be detected.

The present invention relates to a device and method for the detection of carbohydrate antigens, and in particular antigens that are characteristic of microbial/bacterial organisms, such as, for example, the family Streptococcacae, including Group A and Group B Streptococci.

Immunoassays for the detection of organisms, such as from the family Streptococcae, typically rely on the detection of a specific carbohydrate antigen. Antigenic macromolecules (polysaccharides, polypeptides etc.) which append from the outer surface of bacterial walls are specific to the bacterial species, type group or strain. When these antigenic macromolecules are exposed to the corresponding specific antibodies, the antibodies and the antigens bind together to form a precipitin. However, the antigenic molecules must first be released from the cell wall of the organism before they can be detected. A number of methods exist for carrying out such extraction treatments using for example, nitrous acid, ProNase B enzyme, hot formamide or hot HCL. In the case of nitrous acid, as the nitrous acid contacts the exterior walls of the microorganism, the group antigen is released into solution with its antibody specific reactivity intact.

The majority of immunoassay tests that utilise lateral flow technology, for example, utilise nitrous acid for the extraction of the carbohydrate antigen from the organism before contacting the sample containing the extracted antigen with the assay device. Nitrous acid, however, is chemically unstable. It is known only in solution, yet quickly forms nitric acid and nitric oxide in the presence of water. As a result, polysaccharides have conventionally been extracted by the nitrous acid which is formed in a pretreatment step by the reaction between an inorganic nitrate and an aqueous acid, generated immediately prior to antigen extraction. Following the extraction, the solution is then neutralised by the addition of a neutralising agent, such as tris (hydroxymethyl) aminomethane (TRIS) prior to the addition of the resultant solution to the assay device.

Therefore, the extraction of carbohydrate antigen and its subsequent detection would generally be as follows: a swab sample is taken from the back of the throat, for example, and the swab inserted into a vial containing nitrous acid generated by the addition of aqueous solution of sodium nitrite and acetic acid. The swab is subsequently agitated to release the antigens into solution.

Following extraction of the antigen, the solution must be neutralised prior to its addition to a lateral flow or other suitable assay device. In the case of a lateral flow device, the device will typically contain a mobilisable labelled binding reagent capable of binding to the antigen as well as an immobilised binding reagent capable of binding analyte provided downstream from the labelled binding reagent. Thereafter, the antigen may be detected in a conventional manner by formation of an immobilised particulate labelled sandwich binding reagent complex. Such assay devices are disclosed in EP291194.

The method described above, in addition to other prior art methodologies, all require the separate steps of first extracting the antigen by contacting the sample with the precursor nitrite and acid. Both the acid and the nitrite may be contained in solution, or alternatively one of the precursor reactants may be provided in dried form and subsequently contacted with the remaining precursor reactant contained in solution together with the sample. Nevertheless, each of these methodologies require the subsequent step of contacting the sample, following extraction of the antigen, with the lateral flow assay device to detect the desired antigen and which device is provided separately from the sample extraction.

The present invention seeks to alleviate the problems associated with such prior art methodologies and provides a kit and assay device for the detection of antigens or analytes in an organism, wherein the number of treatment steps are substantially reduced and which kit and assay device are therefore particularly easy to use. The test devices according to the invention find particular utility for testing in a nurses or physicians office, a remote or home setting and are therefore particularly useful and suitable for use by non-specialist personnel.

Therefore, according to a first aspect, the invention provides:

a lateral flow assay device for identifying carbohydrate antigens in a biological sample, said device comprising a substrate having

a) a sample receiving zone,

b) an extraction zone for receiving the sample from said sample receiving zone and which extraction zone comprises immobilised or otherwise absorbed therein at least one or more reactants or reagents which when combined react to form an extraction reagent for a desired antigen in said sample,
said sample to be added to said sample receiving zone optionally comprising the remaining reactant required to form said extraction reagent if not present in said extraction zone,

c) optionally, a neutralising agent capable of bringing the pH of the resulting sample to within the operational pH range of the assay, and

d) a detection zone for receiving a labelled specific binding or capture reagent specific for said antigen to be detected.

Lateral flow assay devices are known in the art and generally comprise a porous material with a reagent containing matrix incorporated therein. The sample is permitted to flow laterally from the sample receiving zone downstream to a reaction or detection zone comprising a capture reagent. In the operation of such devices, a test sample is applied to a sample receiving area or zone on the device and the sample moves or flows through the porous material from the application or sample receiving zone to a reaction or detection zone which includes therein an appropriate capture reagent.

The extraction zone may be provided at or downstream from the sample receiving zone.

In one embodiment of the present invention, the precursor reactants are both separately provided in said extraction zone and which when combined by virtue of the movement of the sample through said substrate react to form said extraction reagent. Therefore, when the solution of sample moves laterally through the assay device it will contact each of the reactants in succession such that when they combine they react to form the extraction reagent which can act on the sample to extract any of the desired antigen present. Therefore, the device of the present invention only requires the step of adding the sample to the substrate thus minimising the number of steps required and the amount of sample and reactant manipulation.

The precursor reactants, or reagents, comprise an acid and an acid generating reagent which acid generating agent is able to react with the acid to form a nitrous acid.

The acid is preferably immobilised in the extraction zone of the device by drying, or the like, and is provided separate from the other reagent, such that when the sample moves through the lateral flow assay device, it will come into contact with each of the reagents in succession, both of which will then combine to form the nitrous acid which can then function to extract the antigen from the organism to permit detection.

The immobilised acid may be any suitable acid which is capable of reacting with an acid generating reagent to produce nitrous acid, such as a polycarboxylic acid, a polysulfonic acid, a polystyrenesulphoic acid, a polyphosphoric acid, a polyacrylic acid, and a polymethacrylic acid. Other such acids are known to those of skill in the art.

The acid however, preferably comprises a non-volatile organic, acid such as any of citric acid, malonic acid, phenylacetic acid, oxalic acid, glycolic acid, chloroacetic acid, trichcroacetic acid, fluoroacetic acid, bomoacetic acid, iodaacetic acid, succinic acid, glutaric acid, adipic acid, pimelic acid, suberic acid, benzoic acid, benzene sulfonic acid, p-toluene sulfonic acid, azelaic acid and sebacic acid. However, a polymeric acid, such as polysulphonic acid is particularly preferred and may be provided on said assay device in the form of a resin. An example of a suitable resin is polystyrene.

The acid may be a polymeric acid and may be immobilised by conjugation to particles. Where the acid is conjugated to a particle, the particle size may, advantageously, be chosen so that it is larger than the pore size of the lateral flow assay such that it is unable to move along the length of the porous carrier with the fluid sample.

Alternatively, the acid may form part of the sample fluid carrying matrix such as a sample receiving wick, or the porous carrier itself. This may be achieved, for example, by sulphonation of the material comprising the particular porous matrix.

Any suitable acid generating reagent which is stable in the dry state and capable of reacting with the immobilised acid to produce nitrous acid may be used, such as an alkali metal nitrite.

The acid generating reagent, preferably, comprises a nitrite, which may include any of, inorganic nitrites such as sodium, potassium, lithium, calcium, strontium, barium and silver nitrites, as well as organic nitrites such as butyl and isoamyl nitrites. Sodium and potassium nitrites are preferred while sodium nitrite is most preferred. The concentration of nitrite and organic acid combined in the extraction reagent can be varied widely depending on the particular compounds used, their water solubility and the suspected amount of antigen present in the test sample.

The sodium nitrite may be added to the device in either an aqueous or dry state.

A neutralising agent may also be provided where necessary to bring the fluid sample after it has contacted the extraction zone to within the operational pH range of the assay. The pH range of the assay may vary and will be dependent upon the nature and type of the capture reagents (for example the particular antibody type and whether it is polyclonal or monoclonal) as well as the antigen to be determined. In some circumstances a neutralising agent may not be required should the pH of the resultant sample mixture fall within the operational pH range of the assay.

However, a neutralising agent is preferred as it allows the pH of the reaction mixture to be optimised which in turn allows for optimisation of the assay sensitivity. A typical pH range may be between pH 6-9. The neutralising agent where present is preferably buffered. Many such neutralising buffers are known in the art, although a preferred one is tris (hydroxymethyl) aminomethane (TRIS).

The location of the neutralising agent, where present, will be determined by the operational pH range of the capture reagents. Thus, the neutralising agent may be provided downstream from the extraction zone and upstream from a capture reagent. Alternatively, the neutralising agent may be provided downstream from a first capture reagent and upstream from a second capture reagent. Preferably it is provided within the device in the dry state prior to use.

In one embodiment of the invention, the detection zone may comprise a labelled binding reagent specific for said antigen to be detected, and which binding reagent may be provided in dried or otherwise immobilised form within the detection zone. Alternatively, the labelled specific binding reagent may be added subsequently to the detection zone prior to addition of the sample to be tested. Therefore, the device will, typically, comprise a capture reagent that may, preferably, be first immobilised in the detection zone and which is capable of capturing a particular analyte or antigen of interest. The device may alternatively comprise a mobilisable labelled capture reagent located at a position at or upstream from the detection zone and which is also capable of capturing the analyte or antigen of interest.

In the situation wherein a mobilisable labelled capture reagent is provided in the device prior to use, this reagent is preferably located at a position downstream from the optional neutralising reagent. The mobilisable capture reagent may be provided on and/or in a separate porous carrier arranged so as to be in fluidic contact with and upstream from the porous carrier, such as disclosed by U.S. Pat. No. 6,352,862. The separate porous carrier may be macroporous and may be of a different material to that of the porous carrier. According to one embodiment, the porous carrier is nitrocellulose and the macroporous carrier is chosen from one of a glass fibre or a synthetic polymeric material, such as polypropylene.

Alternatively, the mobilisable capture reagent may be provided in the region of the sample receiving wick or the porous carrier.

The capture reagent may be chosen from any species that is capable of forming a binding pair with the carbohydrate antigen and may be, for example, an antibody or fragment thereof, a lectin, boronic acid or boronic acid derivative. The capture reagent may be a specific capture reagent. The antibody may be either polyclonal or monoclonal.

The presence or amount of analyte in the sample may be determined by the detection of the labelled capture reagent present in the detection zone. The labelled capture reagent may be determined visually or by other suitable means.

The label may be chosen from any suitable label, such as dyed latex spheres or beads or dye imbibed liposomes. The particular label may also comprise a metal sol, such as a gold sol.

A control zone may also be provided in the device according to the invention, which control zone may be provided at a position at or downstream from the detection zone. The control zone serves to indicate whether the test has been carried out correctly. Typically, the control zone comprises a further immobilised capture reagent which is able to capture the labelled capture reagent.

In one embodiment of the device of the present invention, the porous material, preferably comprises a porous wick, which can be made or obtained from any bibulous, porous or fibrous material capable of absorbing liquid rapidly. The porosity of the material can be unidirectional (i.e. with pores or fibres running wholly or predominantly parallel to an axis of the member) or multidirectional (omni directional, so that the member has an amorphous sponge-like structure). Porous plastics material, such as polypropylene, polyethylene (preferably of very high molecular weight), polyvinylidene fluoride, ethylene, vinyl acetate, acrylonitrile and polytetrafluoro-ethylene can be used.

In an alternative embodiment, however, the assay device may comprise one or more microfluidic channels, the dimensions of which are such as to permit a liquid sample to move through the one or more channels by capillary action. The one or more capillary channels may be provided in addition to or instead of the one or more porous or absorbent matrices forming part of the device.

It can be advantageous to pre-treat the carrier material with a surface active agent during manufacture, as this can reduce any inherent hydrophobicity in the carrier material and therefore enhance its ability to take up and deliver a moist sample rapidly and efficiently. Porous sample receiving members can also be made from paper or other cellulosic materials, such as nitro-cellulose.

If desired, an absorbent “sink” can be provided at the distal end of the carrier material. The absorbent sink may comprise of, for example, Whatman 3MM® chromatography paper, and should provide sufficient absorptive capacity to allow any unbound labelled capture reagent to wash out of the detection zone.

As an alternative to such a sink it may be sufficient to provide a length of porous solid phase material which extends beyond the detection zone.

According to a second aspect of the invention, there is provided a kit for identifying carbohydrate antigens in a biological sample, which kit comprises a device as herein before described and means for contacting said sample with said sample receiving area.

Also provided by a further aspect of the invention is a method of producing an assay device according to the invention, which method comprises immobilising in a lateral flow assay device having a sample receiving area, one or more reagents in an extraction zone of said device and which reagents when combined form an extraction reagent for a specific carbohydrate antigen in a sample added to said sample receiving zone, providing a neutralisation buffer downstream of said extraction zone to neutralise the pH of said extraction reagent subsequent to extraction of said antigen from said sample, and upstream from a capture/detection reagent immobilised in said assay device.

In an even further aspect, the invention comprises a method for identifying carbohydrate antigens in a biological sample, which method comprises applying said biological sample to a sample receiving zone of a device as herein before described, and monitoring for the presence of said antigen in said detection zone.

The present invention may be more clearly understood from the following exemplary embodiment which is provided by way of example only, with reference to the accompanying drawings wherein;

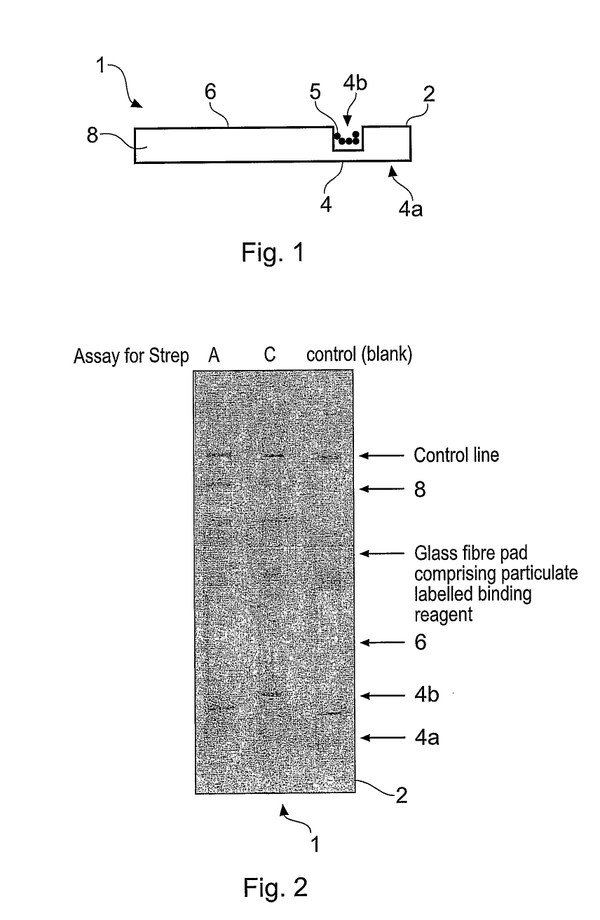

FIG. 1. is an illustration of a device according to the invention.

FIG. 2 is an illustration of the visual results obtained using an assay device of the invention to detect Group A and C Streptococci.

In the device according to the invention as illustrated in FIGS. 1 to 2, there is provided a lateral flow assay device generally indicated by the reference numeral 1, comprising a sample receiving zone or area 2 and an extraction zone 4 for receiving sample moving laterally through the device 1 in the direction from the sample receiving zone to a detection zone 8. In the extraction zone there is provided in a dried form or otherwise immobilised therein an acid generating reagent 4a and an organic acid 4b. A preferred acid generating reagent is sodium nitrite whereas the preferred acid is a polysulfonic acid provided in the form a resin. The polysulfonic acid is provided in a groove or channel 5 cut or etched in the device.

A neutralising buffer 6 is provided downstream of the acid generating reagent and upstream (as defined by the direction of flow in the assay device) of a capture reagent provided in the capture or detection zone 8.

The capture or detection reagent comprises a labelled antibody specific for the antigen to be detected and which can be visualised by virtue of an antigen-antibody complex in the detection/capture zone. Preferably, the antibody is specific for Streptoccal A antigen.

### Experimental

A groove of approx 2 mm wide×3 mm deep was made at a distance of 10 mm from the near end of a porous sample receiving wick of dimensions 48 mm in length×7 mm width×4 mm depth (Filtrona, Richmond Inc, Colonial Heights, Va., USA).

250μ 1M sodium nitrite was applied to one end of the wick and allowed to soak in. 200 μl of 1.6M TRIS pH 8.5 was subsequently applied to the wick at the top end and allowed to soak in, as indicated in FIG. 1. The wick was dried at 50° C. overnight. Approximately 20 mg of sulfonic acid resin (MP 70-90 mesh Novabiochem cat. No. 01-64-0432 1.6 mmole/g) was subsequently packed into the groove.

### Preparation of A Porous Release Matrix Comprising A Mobilisable Particulate Labelled Binding Reagent

Anti Strep A carbohydrate antibody (Biotech Inc.) was absorbed onto 400 mm blue latex particles (Duke Scientific, USA) at a concentration of 30 μg/ml (0.5% latex solids) was prepared containing the freeze dried latex particles coated with the antibody. The antibody sensitised latex particles were infused into a glass fibre pad of dimensions 20 mm×8 mm×1.2 mm pad having an open structure (Sintair, USA) and freeze dried.

The wick containing the dried extraction reagents was subsequently placed on top of the prepared glass fibre pad.

### Preparation of the Porous Carrier Comprising An Immobilised Capture Reagent

Anti Strep A polyclonal antibody was deposited by means of a microprocessor-controlled microsyringe onto an 8 mm wide porous nitrocellulose carrier strip (Schleicher and Schuell based 8μ porosity) at a concentration of 1.9 mg/ml and at a flow rate of 0.8 μl/mm.

The antibody was applied in the form of a stripe oriented perpendicular to the flow of liquid sample at a detection zone located downstream from the labelled antibody and the antibody was subsequently blocked with 1% (w/v) polyvinyl alcohol/3% (w/v) sucrose to immobilise it to the porous carrier.

### Construction of the Device

Lateral flow carrier devices were constructed as shown in FIG. 1 using the materials as prepared above. A porous sample receiving wick containing the dried extraction reagents was placed on top of a glass pad containing the freeze dried antibody sensitised latex particles coated with anti Strep A polyclonal antibody.

The glass pad was subsequently placed in contact with the nitrocellulose porous carrier such that liquid sample applied to one end of the porous sample receiving wick would be capable of permeating into the glass fibre pad and along the nitrocellulose porous carrier.

A further anti mouse antibody was immobilised at a location downstream from the immobilised capture reagent which functioned as a control zone. Finally an absorbent sink pad was placed in contact with the distal end of the nitrocellulose strip.

A further device was also constructed for the detection of Streptococcus C as described above except the anti Strep A antibodies were replaced by anti-Strep C antibodies.

Samples of Strep A and C were respectively diluted to 10e9 cfu/ml in water. The bacterial suspensions (100 μl) were applied to the near end of the device (sodium nitrite end), followed by 1000 μl of water (to move the liquid through the device).

As a control the test was repeated except that 100 μl of water containing no bacteria was applied to a device. The results of the tests are shown by FIG. 2. As may be seen from FIG. 2, the presence of blue latex sensitised antibodies may be observed at the detection zone indicating the presence of Strep A and C carbohydrate antigen.

Thus results show that the respective carbohydrate antigens were released from the bacteria by nitrous acid generated by re-hydration of the reagents contained within the device, and that these antigens were then able to be specifically detected in the subsequent immunoassay.

1. A lateral flow assay device for identifying carbohydrate antigens in a biological sample, said device comprising a substrate having
a) a sample receiving zone,
b) an extraction zone for receiving the sample from said sample receiving zone and which extraction zone comprises immobilised or otherwise absorbed therein at least one or more reactants and reagents which when combined react to form an extraction reagent for a desired antigen in said sample,
said sample to be added to said sample receiving zone optionally comprising the remaining reactant required to form said extraction reagent if not present in said extraction zone,
c) optionally a neutralising agent capable of bringing the pH of the resulting sample to within the operational pH range of the assay and,
d) a detection zone for a labelled specific binding or capture reagent for said antigen to be detected.

2. A device according to claim 1 wherein said extraction zone comprises both said precursor reactants that when combined react to form said extraction reagent.

3. A device according to claims 1 or 2, wherein said antigen to be detected is a Streptococcal antigen.

4. A device according to claim 3, wherein said Streptococcal antigen is Streptococcal A antigen.

5. A device according to any preceding claim wherein said extraction reagent is a nitrous acid, each of said precursor reactants being an acid and a nitrite and which when combined form the nitrous acid.

6. A device according to claim 5, wherein said acid is a sulfonic acid in the form of a resin and said nitrite is sodium nitrite.

7. A device according to any preceding claim wherein said substrate comprises a lateral flow porous carrier.

8. A device according to any of claims 1 to 6 wherein said substrate comprises microfluidic channels the dimensions of which are such as to allow a liquid sample move there through by means of capillary action.

9. A device according to any preceding claims wherein said labelled specific binding reagent is immobilised at or upstream of said detection zone.

10. A device according to any of claims 1 to 9, wherein said labelled specific binding reagent is added to said substrate together with or after addition of said sample.

11. A device according to any of claims 1 to 9 wherein said substrate comprises a mobilisable labelled specific capture reagent and provided at a position downstream from the optional neutralising agent.

12. A device according to claim 11, wherein the mobilisable capture reagent is provided on and/or in a separate porous carrier arranged so as to be in fluidic contact with and upstream from the porous carrier.

13. A device according to claim 12, wherein said separate porous carrier is macroporous and may be of a different material to that of the porous carrier.

14. A device according to any preceding claim wherein said labelled specific binding reagent is labelled antibody.

15. A device according to any preceding claim where said label is an enzyme label or a specific dye.

16. A device according to any preceding claim, wherein said sample receiving zone comprises a sample receiving wick made of any bibulous, porous or fibrous material capable of absorbing liquid sample.

17. A device according to any preceding claim, wherein the porous carrier is a nitrocellulose carrier.

18. A device according to claim 17, wherein said porous carrier is nitrocellulose and the macroporous carrier is selected from any of glass fibre of a synthetic polymeric material such as polypropylene.

19. A device according to any preceding claim, wherein said substrate comprises an absorbent sink at the distal end thereof.

20. A device according to any preceding claim further comprising a control zone at or downstream from the detection zone and which comprises a further immobilised capture reagent that is able to capture the labelled capture reagent.

21. A kit for identifying carbohydrate antigens in a biological sample, which kit comprises a device according to any preceding claim and means for contacting said sample with said sample receiving area.

22. A method of producing an assay device according to any of claims 1 to 20, which method comprises immobilising in a lateral flow assay device having a sample receiving area, one or more reagents in an extraction zone of said device and which reagents when combined form an extraction reagent for any carbohydrate antigen in a sample added to said sample receiving zone, providing a neutralisation buffer downstream of said extraction zone to neutralise the pH of said extraction reagent subsequent to extraction of said antigen/analyte from said sample, and upstream from a capture/detection reagent immobilised in said assay device.

23. A method according to claim 22, wherein said method comprises pretreating said substrate with a surface active agent.

24. A method for identifying carbohydrate antigens in a biological sample, which method comprises applying said biological sample to a sample receiving zone of a device according to any of claims 1 to 20 and monitoring for the presence of said antigen in said detection zone.
